(19) United States
(12) Patent Application Publication
(10) Pub. No.: US 2010/0279409 A1
(43) Pub. Date: Nov. 4, 2010

(54) METHOD FOR MODIFYING CELLUAR IMMUNE RESONSE BY MODULATING ACTIVIN ACTIVITY

(76) Inventors: Neil Robson, Edinburgh (GB); Eugene Maraskovsky, Victoria (AU); David Phillips, Victoria (AU); Jonathan Cebon, Heidelberg (AU)

Correspondence Address: FULBRIGHT & JAWORSKI, LLP, 666 FIFTH AVE, NEW YORK, NY, 10103-3198, US

(21) Appl. No.: 12/733,670

(22) PCT Filed: Sep. 11, 2008
(86) PCT No.: PCT/US08/10607

§ 371 Date: Jul. 16, 2010

Related U.S. Application Data
(60) Provisional application No. 60/993,620, filed on Sep. 13, 2007.
(60) Provisional application No. 61/132,834, filed on Jun. 23, 2008.

(51) Int. Cl. C12N 5/0783

(52) U.S. Cl. 435/375

(57) ABSTRACT

The invention relates to methods for modifying a cellular response, such as a CD8⁺ T cell or NK cell response, by adding an activin modulator in an amount sufficient to modulate activin production. By modulating this production, the cellular response is itself modulated.

### FIELD OF THE INVENTION

This invention relates to regulation of cellular function via inhibition of activin molecules. More particularly, it relates to the inhibition of activin as a way to enhance specific immune responses.

### BACKGROUND AND PRIOR ART

The activin family of molecules are well known and have been studied extensively. Activin-A, for example, is a homodimer of activin β_{A} subunits, and was first described, by Ling, et al., Nature, 321:779-782 (1986), as a reproductive factor which accentuates release of follicle stimulating hormone. It, as with other activins, is a member of the transforming growth factor-β (“TGF-β”) superfamily of cytokines, sharing Smad intracellular signaling proteins with TGF-β. See Shi, et al., Cell, 113:685-700 (2003). Signaling for activin-A does occur through separate and distinct serine threonine kinase receptor subunits, with release into circulation during acute systemic inflammation occurring via different pathways. See, e.g., Phillips, The Activin/Inhibin Family, vol. 2, edition 4 (London, Academic Press, 2003). Specifically, activin-A signals through heteromeric receptor complexes of both type I (ALK-2, 4, or 7) and type II (Act RITA and Act RIIB) receptors. This versatile molecule is known to have pivotal roles in, inter alia, induction of apoptosis, exacerbation of rheumatoid arthritis embryonic stem cell renewal and pluripotency, and differentiation of erythroid lineage cells. (Xiao, et al., Stem Cells, 24:1476-1486 (2006); and Jiang et al., Stem Cells, 25:1940-1953 (2007)). Activin-Smad signaling pathways have been shown by Rosendahl, et al., Int. Immunol., 15:1401-1414 (2003), to be activated at distinct maturation stages of thrombosis in mice. Additional examples of Activin's pleiotropic nature can be found in its pro and anti-proliferative effects on tumor cells (Yamashita, et al., Cancer Res., 50:3182-3185 (1990); Brudette, et al., Cancer Res., 65:1968-1975 (2005), and Panapoulou, et al., Cancer Res., 65:1877-1886 (2005)); pancreatic fibrosis (Sulyok, et al., Mol. Cell Endocrinol., 225:127-132 (2004)); rheumatoid arthritis (Ota, et al., Arthritis Rheum., 48:2442-2449 (2003); and diabetes (Li, et al., Diabetes, 57:6508-615 (2004)).

Hedger, et al., Cytokine, 12:595-602 (2000), have shown that activin-A can either inhibit or stimulate rat thymocyte growth and differentiation.

A number of researchers have noted that the biological activity of activin-A is controlled at many levels, including interaction with follistatin. Representative are Nakamura, et al., Science, 247:836-838 (1990); Nakamura, et al., J. Biol. Chem., 266:19432-19437 (1991); Mather, et al., Endocrinology, 132:2732-2734 (1993); and Phillips, et al., Front Neuroendocrinol, 19:287-322 (1988). The mechanism of interaction, in brief, involves the binding of two follistatin molecules to one of activin, resulting in the burying of 1/3 of activin-A's residues, which in turn antagonizes the binding to both types of receptors. See Thompson, et al., Dev. Cell, 9:535-543 (2005). Jones, et al., Mol. Cell. Endocrinol, 225:119-125 (2004), have proposed that follistatin's involvement in inflammatory processes is part of a short feedback loop which modulates and suppresses activin-A. To elaborate briefly, during inflammation, systemic release of follistatin occurs after activin-A release, and follistatin is believed to modulate and to suppress activin's effects.

Dendritic cells are well known as being involved in the formation of “sentinel networks” within the body, sampling the microenvironment and responding to pathological challenge via any of a number of pattern recognition receptors. See, e.g., Bauer, et al., J. Immunol., 166:5000-5007 (2001); Gallucci, et al., Curr. Opin. Immunol., 13:114-119 (2001); and, L'Ositani, et al., J. Exp. Med., 191:1661-1674 (2000). Pathogen encounter leads to maturation of the dendritic cells, which in turn leads to profound alterations in function. Caux, et al., J. Exp. Med., 180:1263-1272 (1994); Inaba, et al., J. Exp. Med., 188:2163-2173 (1998); Turley, et al., Science, 288:522-527 (2000); Robson, et al., Immunology, 109:374-383 (2003); and, Luft, et al., Blood, 104:1066-1074 (2004) have all commented on the processes of how antigen uptake is reduced, antigen processing is enhanced, and pro-inflammatory mediators released. Luft, et al., supra, have also discussed in some detail, the mechanisms of paracrine and autocrine signaling involved.

Production of cytokines and chemokines by dendritic cells (“DC” hereafter) can be induced by, e.g., CD40L and TLR agonists (e.g., LPS, intact bacteria).

The appropriate release of these molecules, either by DCs, or other neighboring cells, is critical in the induction and moderation of inflammation, the recruitment of innate effectors, and the regulation of T cell cytokine production. See, e.g., Romani, et al., Int. Rev. Immunol., 6:151-161 (1990); Heufter, et al., J. Exp. Med., 176:1221-1226 (1992); McWilliam, et al., J. Exp. Med., 184:2429-2432 (1996); and, Sporri, et al., Nat. Immunol., 6:163-170 (2005). Many of the molecules produced by DCs, at the epicenter of infection and inflammation, including IL-6, IL-8, IL-10, and IL-12p70 have pleiotropic effects, ranging from enhancement or inhibition, depending on context and the targeted cells. Levy, et al., Proc. Natl. Acad. Sci. USA, 87:3309-3313 (1990); Cavallo, et al., Clin. Exp. Immunol., 96:1-7 (1994); and Mowat, et al., Clin. Exp. Immunol., 99:65-69 (1995), have all discussed how uncontrolled release of these substances within this microenvironment can result in inappropriate T and/or B cell responses, with resulting immune pathology. To this end, the immune system has evolved in such a way that the expression of mediators is coordinated to attenuated exaggerated or inappropriate responses, to minimize tissue damage and immune pathology. The range of molecules expressed as a result of this include PGE₂, ATP, and TGF-β. See, e.g., Strassmann, et al., J. Exp. Med., 180:2365-2370 (1994).

The known facts regarding activin-A and follistatin, and their role in inflammation, suggested that they may have a role in DC function.

There is a well-known and well documented interaction between DCs and natural killer (NK) cells. See, e.g., Degli-Esposti, et al., Natl. Rev. Immunol., 5:112-124 (2005). NK cells are innate, immune cells which recognize and kill virus infected or tumor cells. See, Kiessling, et al., J. Exp. Med., 143:772-780 (1976); Smyth, et al., Nat. Rev. Cancer, 2:850-861 (2002); and Andomou, et al., Immunol. Rev., 214-234-250 (2006). They also have the potential to play an important role in regulating both innate and adaptive immunity via direct interaction with DCs in the lymph nodes, or with inflamed tissues, via IFN-γ production. See, e.g., Degli-Esposti, et al., supra. DC derived, IL-12 p70 has been recognized as a potent co-factor for enhancing NK cell toxicity and IFN-γ production, which in turn is responsible for the initial shaping of T helper type 1 immunity (D'Andrea, et al., J. Exp. Med., 276:1387-1398 (1992); and Martin-Fintecha, et al., Nat. Immunol., 5:1260-1265 (2004).

It has now been found that DCs, including human DCs, respond to activin-A, and that autocrine activin-A production by DCs can attenuate their pro-inflammatory potential, and their T cell stimulatory capacity, including the expansion of antigen specific, CD8⁺ T cells.

It has also been found that NK cells express activin-A receptors, and activin-A attenuates NK cell IFN-γ production, proliferation and phenotypic maturation, but has no impact on the ability of the NK cells to kill tumor cell targets.

Hence, it is one aspect of the invention to show the interaction of activins and their inhibitors has a potent effect on immune cells, such as NK cells and DCs.

How this is accomplished will be seen in the disclosure which follows.

### DETAILED DESCRIPTION OF PREFERRED EMBODIMENTS

### Example 1

The relevance of the activin system to human DC populations was assessed by studying the expression patterns of type I and type II human activin receptors as well as activin-A subunit mRNAs.

To do this, dendritic cells were either generated from CD14⁺ cells, or purified from blood.

With respect to the first approach, CD14⁺ cells were isolated via positive selection using magnetic beads coated with anti-CD14⁺ antibodies, using standard techniques. The CD14⁺ cells were then cultured with GM-CSF and IL-4 for 6-7 days, with cultures being maintained in RPMI 1640 supplemented with 20 mM HEPES, 60 mg/L penicillin, 12.5 mg/L streptomycin, 2 mM L-glutamine, 1% non-essential amino acids, and 10% heat inactivated fetal calf serum.

Once cells were obtained, their RNA was isolated, using standard methods, and cDNA was synthesized therefrom, also via standard methods.

Standard RT-PCR was carried out, using primers specific for the RNA in question.

Initial experiments showed that MoDCs expressed activin receptor type I and II, and activin βA subunit mRNA.

### Example 2

In follow up experiments, the kinetics of gene expression described in Example 1 were studied, by stimulating MoDCs with either of trimeric CD40L or LPS, via quantitative real time PCR, or “qRT-PCR,” using standard methods and primers specific for activin βA and the activin receptor molecules ALK-2, and ALK-4. The primers differed from those used in the RT-PCR work, described supra. Stimulus was accomplished with 2 μg/ml of CD40L trimer, or 100 ng/ml of E. coli derived LPS for anywhere from 2-24 hours. As a control, MoDCs were cultured in GM-CSF plus IL-4 for the same length of time.

The results showed the ALK-2 mRNA was only detected at very low levels in the MoDC cultures. Early in the culture period, both CD40L trimers and LPS downregulated ALK-4 mRNA, but when the stimulation was continued for longer periods it increased, to a point where it was significantly higher than controls after 24 hours.

Both CD40L trimer and LPS stimulation upregulated activin RIIA after 2 hours, after which it decreased to background levels by 6 hours. In contrast, expression of activin RIIB mRNA was markedly different, with early down regulation, followed by increases over time, when stimulated by CD40L, while LPS continued to suppress expression.

With respect to activin βA subunit mRNA, large increases resulted after as little as 2 hours, with a peak at 4-6, followed by a decrease at 24 hours.

When CD123⁺, and CD1c⁺ PBDCs were tested, it was found that these cell types expressed low levels of ALK-2 and ALK-4 type I receptors, with more prominent expression of constitutively expressed activin RIIA receptor. Culture alone, without stimulus, induced activin βA subunit mRNA in CD123⁺ and CD1c⁺ PBDCs.

The results suggest that there is a very complex level and pattern of activin receptor and activin-A gene regulation in MoDCs response to stimulation by CD40L or LPS.

### Example 3

These experiments were designed to determine if upregulation of activin βA subunit gene expression translated into increased secretion of activin-A protein.

Human MoDCs were co-cultured, in a medium containing both GM-CSF and IL-4, with one of (i) intact E. coli, (ii) CD40L, (iii) a TLR ligand (LPS, R848, Poly I:C, or Pam₃Cys, which bind, respectively, to TLR4, 7/8, 3, and 2/6), or (iv) one of the inflammatory mediators PGE₂ or ATP. Culture was carried out for from 2-72 hours. Supernatants were collected, and assayed for activin-A, using standard methods.

Intact E. coli induced the highest level of activin-A secretion, which was consistent with the results obtained for upregulation of activin βA subunit mRNA.

Signaling through CD40 or TLR4 produced very similar kinetics and magnitude of activin-A release, with significant activin-A levels detected at 6 hours, peaking at 48 hours. MoDCs do express receptors for PGE₂ and ATP, neither of these stimulated activin-A release above the levels observed in unstimulated MoDC cultures. R848 and Poly I:C stimulated MoDCs to produce significant amounts of activin-A after 6 hours of stimulation. Pam₃Cys, in contrast, did not.

### Example 4

The results showing that MoDCs secreted large amounts of activin-A in response to the various stimuli suggested examining other cell types to determine whether or not they produced major amounts of activin-A protein.

CD1c⁺ and CD 123⁺ PBDC cell population were stimulated, for 2, 4, and 24 hours, with one of intact E. coli, CD40L trimer, or a TRL ligand (LPS, R848, Poly I:C). Stimulation took place as described. Controls were also as described supra.

Activin-A was detected at low levels in the culture supernatants, at 4 hours, and peaked at 24 hours. It was striking to note that unstimulated CD1c⁺ PBDCs produced levels of activin-A that were equivalent to those levels seen following stimulation. This is consistent with the previous observation that these cells mature spontaneously when cultured in vitro. The CD123⁺ PBDCs spontaneously upregulated activin βA subunit mRNA upon in vitro isolation, but produced very low levels of activin-A protein, regardless of stimulation.

T cell, B cell, and NK cell samples were also tested. In contrast to DCs and to activated murine splenic CD4 T cells, activated, human CD4⁺ T cells which had been purified from blood, did not produce activin-A, nor did CD8⁺ T cells, B cells, or NK cells, when tested over the 24 hour period.

### Example 5

It has been shown by others that stimulation by materials such as those tested herein (CD40L, LPS, R848, Poly I:C), powerfully induces the maturation of MoDCs and cytokine secretion. As noted supra, it was observed for the first time that, under these conditions, MoDCs very rapidly secrete large quantities of activin-A. This suggested tests to determine if the activin-A that was produced resulted in paracrine or autocrine signaling, or both. To test this, immature MoDCs were cultured for 4 hours with GM-CSF plus IL-4, as a control, or CD40L (2 μg/ml), LPS (100 ng/ml), R848 (1 μg/ml), or Poly I:C (10 μg/ml). After 4 hours, the DCs were lysed, the proteins were extracted, and Western blotting was carried out to measure Smad 2/3, phosphorylated-Smad 2, and β-actin. The Western blotting was carried out using commercially available antibodies, and standard protocols.

The increased levels of Smad 2/3 and phosphorylated-Smad 2 found in stimulated MoDC populations, taken with increased expression levels of activin receptors (shown supra), suggests that enhanced autocrine stimulation was the most likely result, with paracrine signaling resulting therefrom.

### Example 6

The early autocrine/paracrine release of activin-A by MoDCs was viewed as a possible potentiating feedback loop to further activin-A production. To test this, immature MoDCs were cultured with 2 μg/ml of CD40L, together with increasing concentrations of follistatin (0-400 ng/ml). Supernatants were collected over a 6 hour period and activin-A was qualified in a standard ELISA.

Over the first 2-4 hours, increasing amounts of follistatin resulted in dose dependent decreases in activin-A production. At 6 hours, however, even 400 ng/ml was unable to abrogate the level of activin-A production, suggesting either that the levels of activin-A produced by 6 hours were no longer neutralizable by follistatin, or that the signaling cascade that had been initiated could not be reversed.

### Example 7

Prior studies had shown that, in an in vivo LPS challenge model, activin-A is released into the circulatory system rapidly, and can be detected prior to IL-6, and just prior to TNF-α release. The data described supra, showing that all of live E. coli, bacterial LPS, and trimeric CD40L induce secretion of large amounts of activin-A by MoDCs, suggested that this release may influence subsequent production of IL-6 by these MoDCs. This was tested by combining MoDCs with trimeric CD40L, or LPS, at a fixed concentration, together with varying concentrations of follistatin (0, 20, and 100 ng/ml) MoDCs were combined with GM-CSF and IL-4 as a control. Supernatants were collected after 4 hours, 24 hours, and 48 hours of culture, and then assayed for IL-6 via a standard ELISA.

It was found that LPS stimulated production of large amounts of IL-6 by 24 hours; however, follistatin did not influence its production. This was also the case for IL-8 and IL-12p70 production by the MoDCs.

The results for CD40L, in contrast, demonstrated a dose dependent effect, i.e., as follistatin concentration increased, so too did IL-6 production, to levels that were also seen with CD40L and E. coli.

It is important to note that the addition of follistatin alone did not induce MoDC cytokine production.

Further experiments were then carried out, using trimeric CD40L or concentrations of LPS and follistatin that were even higher (1 ng/ml-150 ng/ml for LPS, and 400 ng/ml for follistatin). The experiments were carried out as described in the first set of experiments, and again, antagonizing activin signaling with follistatin dramatically enhanced the CD40L—mediated production of IL-6, but not LPS mediated IL-6 production, at any dose.

When qRT-PCR was carried out, a 15 fold increase in IL-6 mRNA expression was observed 2 hours after MoDC stimulation and the 400 ng/ml dose of follistatin, with a peak at 4 hours, (30 fold), with a decrease over 6-24 hours.

### Example 8

The experiments of the preceding examples were expanded in order to more fully investigate the effect of MoDC cytokine secretion.

Supernatants were screened, via standard ELISAs, for a large array of cytokines and chemokines, including pleiotropic IL-10.

It was found that antagonizing activin-A with follistatin did not impact E. coli mediated IL-10 secretion; however, it did enhance the specific CD40L mediated IL-10 secretion by the same population of MoDCs. When follistatin was added, it enhanced IL-10 secretion by the MoDCs in a dose dependent manner, which correlated with increased IL-10 mRNA expression. This peaked at 4 hours, and waned at 24.

### Example 9

The prior examples demonstrated that inhibition of activin-A during CD40L stimulation profoundly enhanced IL-6 and IL-10 production in MoDCs. In view of this, the experiments were extended to investigate the impact, if any on IL-12p70, a potent TH1 cytokine, and pro-inflammatory TNF-α. The experiments were carried out in the same way as the prior experiments were.

The rate limiting subunit in formation of IL-12p70 is IL-12p35. The kinetics of the subunit's expression differed markedly as compared to the kinetics of IL-6 expression, the addition of follistatin to neutralize activin-A resulted in at least 15 fold greater levels of IL-12p35 expression at 24 hours, as compared to the levels of expression which were obtained following stimulation with CD40L alone.

Real time PCR (“RT-PCR”) showed that upregulation in IL-12 gene expression, in turn, resulted in dose dependent enhancement in IL-12p70 protein secretion in the presence of follistatin.

As was the case with IL-6 production, follistatin alone, without any other stimulus, did not induce IL-12p70 secretion by MoDCs. TNF-α secretion mirrored the production of IL-12p70, with dose dependent enhancement peaking at 24 hours.

In parallel experiments, the major type 1 activin receptor, ALK-4, was inhibited using SB431542, a well known inhibitor of that molecule and it had similar enhancing effects in CD40L mediated cytokine production by MoDCs, as was seen with follistatin.

These experiments all lead to the conclusion that, while all of CD40L, LPS, and live E. coli induce large amounts of activin-A, inhibition of activin-A signaling with follistatin does not appear to modulate MoDCs production of IL-6, IL-10, or IL-12p70 in response to LPS or E. coli, but does have a potent effect on CD40L mediated cytokine secretion.

### Example 10

It is well known that chemokines, as well as cytokines, play an integral part in the processes involved in recruitment of leukocyte effectors, and the shaping of immune responses. Given the role of activin-A in regulating and modulating CD40L induced chemokine secretion, as shown in the prior examples, it was of interest to explore what role activin-A might have in CD40L induced chemokine secretion.

MoDC culture supernatants were screened following 24 hours of culture, using controls, and increasing concentration of follistatin, as described supra. Assays were carried out, following standard methods, for IL-12p70, TNF-α, IL-8, IP-10, RANTES, and MCP-1.

As with the cytokines antagonizing CD40L induced activin-A production with follistatin substantially enhanced production of IL-8, RANTES, and MCP-1 by MoDC. Taken together, one has clear evidence of a previously undescribed regulatory role of activin-A in CD40L mediated cytokine and chemokine secretion by dendritic cells.

### Example 11

These experiments investigate if activin-A regulated dendritic cells' ability to induce adaptive T cell responses. First, CD8⁺ T cells that had been purified via standard methods and which were shown to respond to anti-CD3/CD28 treatment by upregulating type I and type II activin receptors.

Autologous assays were then carried out, in which CD8⁺ T cells were co-cultured with MoDCs that had been pulsed with chemically inactivated influenza virus, and the saponin based, commercially available adjuvant ISCOMATRIX. ISCOMATRIX effectively targets antigens to class I MHC processing pathways in dendritic cells, and thus allows for efficient class I MHC cross presentation of peptide antigens. This adjuvant is a derivative of a adjuvant known as ISCOM, which is a saponin based adjuvant, shown to be safe, well tolerated, and able to induce strong antibody and T cell responses, in animals and humans. See, e.g., U.S. Pat. No. 6,351,697, and PCT application WO 96/11711, both of which are incorporated by reference. In essence, ISCOM vaccine comprises saponin, cholesterol and antigen wherein the antigen is associated with the saponin:cholesterol complex via hydrophobic interaction. ISCOMATRIX vaccine comprises the same components but the antigen is not associated by hydrophobic interactions. Also, see Barr, et al., Immunol Cell Biol., 74:8-25 (1996) and Ennis, et al., Virology, 259:256-261 (1999). The combination of the immature MoDCs, the inactivated influenza particles, and the adjuvant were pulsed for 6 hours, prior to washing and co-culture of 2×10⁴ MoDCs with 2×10⁵ purified, autologous CD8⁺ T cells, either with or without 400 ng/ml follistatin. The base culture medium contained 10 U/ml IL-2 and 20 ng/ml GM-CSF.

After nine days, the cultures were counted, and restimulated by co-culture with HLA-A2 restricted influenza matrix peptide pulsed T2 cells, together with anti-CD107a antibody, in the presence of BFA (brefeldin-A).

Inhibition of activin-A, using follistatin, significantly increased both the percentage and total number of influenza matrix specific CD8⁺ T cells which displayed effector function, as determined by antigen specific IFN-γ secretion, and lytic granule exocytosis, determined via CD107a expression.

These results show that activin-A not only regulates DC cytokine output via autocrine/paracrine mechanisms, but also limits the capacity of the DCs to expand antigen specific CD8⁺ T cell effectors. It is very important to note that the regulatory effects of activin-A were antagonized by contact with follistatin, thus revealing the full T cell stimulatory potential of antigen loaded dendritic cells.

### Example 12

The experiments were designed to determine if activin-A directly regulated in vitro CD8⁺ T cell activation and expansion.

To test this, CD8⁺ T cells were purified and labeled with carboxy fluorescein succinimidy/ester (“CFSE” hereafter). These cells were cultured in wells, at 1×10⁵ cells/well, in standard medium supplemented with 20 U/ml of IL-2, either with or without anti-CD3/CD28 bead stimulation, in the presence of increasing doses of commercially available, recombinant activin-A (0, 10, and 100 ng/ml), for 24 or 72 hours. Cell division was assessed over time by flow cytometry and IFN-γ production via a standard ELISA.

Even at activin-A doses 10 times higher than those produced by MoDCs, activin-A did not regulate either CD8⁺ T cell division or the ability to generate IFN-γ.

These results suggested that activin-A may not directly regulate T cells; rather its influence may be via regulation of DC function.

### Example 13

These experiments show that activin-A has a potent ability to suppress peptide specific CD8⁺ T cell responses in vivo. C57/B6 mice were injected, subcutaneously, with one of PBS, 100 ng of activin-A, or a premixed combination of 100 ng of activin-A and 400 ng follistatin. The injections were administered 1 hour before, and then 24 and 48 hours after intraperitoneal infection with 10⁶ plaque forming units (PFU) of live influenza virus. Seven days later, the animals were sacrificed, spleens removed, and “ip washes” were collected. Spleen cells were separated out and these, plus the cells of the IP wash, were restimulated, in vitro, for 6 hours with CTL peptides specific for the acidic polymerase, or nucleoprotein immunodominant determinants within the virus.

BFA was added for the last 4 hours of the culture, before an ICS assay was performed. When the percentage of peptide specific, CD8⁺ IFN-γ⁺ T cells was determined by flow cytometry, it was found that activin-A had suppressed the in vivo, peptide specific CD8⁺ T cell response, confirming the importance of activin-A in regulating adaptive T cell immunity.

### Example 14

The results set forth supra suggested extension of the experiments to additional cell types. These experiments involve work in NK cells.

Human NK cells were highly purified, following standard methods; and were either lysed immediately or cultured for 20 hours in medium that had been supplemented with 20 U/ml IL-2, either with or without 10 ng/ml of IL-12, or with or without the toll like receptor 3 ligand Poly I:C, 5×10⁵, lysed immediately, and then assayed for ALK-4 (an activin type I receptor), and RITA (an activin type II receptor), via qRT-PCR. In parallel, 1×10⁵ highly purified NK cells were cultured in medium supplemented with 20 U/ml IL-2, with or without 50 ng/ml of recombinant human activin-A. Cells were harvested after 4 hours, and Western blotting was carried out for Smad 2/3 and β-actin.

The results indicated that the highly purified populations of cells expressed mRNA for activin I type (ALK4) and II (RIIA) receptors. The ALK4 mRNA levels were increased through culture in media supplemented with IL-2 only, wherein specific stimulation, with IL-2 and IL-12, plus the TLR-3 ligand poly I:C resulted in increased expression of activin RIIA.

When recombinant activin-A was added, to ex vivo purified NK cells, enhanced intracellular Smad 2/3 levels were seen within 4 hours.

The totality of these results show that human NK cells do express receptors for, and respond to activin-A. This suggested further investigations into what role activin-A might have, in NK cell function.

### Example 15

A total of 1×10⁵ highly purified human NK cells were cultured for 20 hours in medium supplemented with 20 U/ml IL-2, and 10 ng/ml IL-12. Either human recombinant activin-A, or TGF-β was added to the cultures, at concentrations of 10, 50 and 100 ng/ml. Supernatants were then tested for IFN-γ via ELISA, using standard methods.

The results indicated that activin-A was as effective as TGF-β in suppressing the production of IFN-γ by stimulated cells. Via analysis of markers associated with apoptosis (caspase) or cell death (propidium iodide), it does not appear that either of these mechanisms is responsible for suppression of IFN-γ production.

### Example 16

In these experiments 1×10⁵ highly purified human NK cells were cultured as above, but were cultured with Poly I:C (10 μg/ml), or LPS (100 ng/ml), with or without 10, 50, or 100 ng/ml of recombinant human activin-A. In some cases, the known compound “SB431542,” which blocks activin-A binding to receptors was added. IFN-γ was then measured via ELISA, or by intracellular cytokine staining after 16 hours of culture, via addition of 1 μg/ml BFA, to prevent cytokine release.

The results indicated that activin-A suppressed IFN-γ production even when cells were stimulated with Poly I:C, and this was not due to a decrease in the number of IFN-γ producing cells, which suggests suppression of the amount of production per cell.

In parallel, flow cytometry was carried out to determine the effect of activin-A on the IL-2 receptor alpha chain, “CD25,” and CD56, which is an indicator of NK cell phenotypic maturation The results indicated that upregulation of CD25 and downregulation of CD56 were suppressed by activin-A, suggesting suppression of NK phenotypic maturation.

### Example 17

In these experiments, 1×10⁵ highly purified human NK cells were labeled with CFSE, and cultured for 5 days in medium supplemented with 20 U/ml IL-2 and 10 ng/ml IL-12, either with or without Poly I:C or LPS, either with or without 100 ng/ml of human recombinant activin-A. Cellular division was assessed by determining loss of CFSE intensity using known, flow cytometric methods.

In both cases (Poly I:C and LPS), activin-A very strongly suppressed the division and proliferation of the NK cells.

In follow up experiments, the impact of follistatin on activin-A was tested, in the context of mature monocyte derived dendritic cells.

The dendritic cells were obtained by culturing CD14⁺ monocytes with GM-CSF and IL-4, for 6-7 days, which are standard culture conditions. A sample of 5×10⁴ MoDCs were co-cultured with 1×10⁵ NK cells, either with or without 400 ng/ml follistatin. As controls, samples of each type of cell were cultured individually.

As the MoDC cells produce activin-A under the culture conditions used, none was added.

IFN-γ production was measured, as described supra.

The results indicated that the amount of IFN-γ produced in the absence of follistatin increased markedly with follistatin present, indicating that activin-A had been inhibited.

### Example 18

Since it is known that NK derived IFN-γ is a potent factor in shaping Th1 immunity, experiments were carried out to determine if activin-A produced by dendritic cells (“DCs”) could regulate NK cell production of IFN-γ directly.

To test this, 5×10⁴ immature MoDCs were cultured, in 96 well-plates, in standard media supplemented with 20 U/ml IL-2 and 20 ng/ml GM-CSF only, or with the addition of 1×10⁵ autologous NK cells. In addition, the cells were stimulated with IL-12 and poly I:C. In addition, 400 ng/ml of follistatin was added to cultures in order to neutralize activin-A.

After 24 hours, supernatants were collected, and assayed for IFNγ via a standard ELISA.

The results indicated that IFN-γ was not detected in cultures of immature MoDCs without NK cells, even in the presence of IL-12 and poly I:C, nor did mixed cultures of immature MoDCs and NK cells produce IFN-γ. The combination of co-cultured MoDCs and NK cells, with IL-12 and poly I:C resulted in IFN-γ production by NK cells. More significantly, the addition of follistatin, activin-A's natural antagonist, resulted in significant increases in IFN-γ production.

Previously, it had been shown that NK cells do not produce activin-A, and the addition of follistatin to NK cells stimulated with cytokines, had no impact on their IFN-γ production.

### Example 19

The next set of experiments were designed to determine the effect of activin-A on NK cell IFN-γ production, gene regulation, and cell viability.

The impact of activin-A on production of IFN-γ by NK cells, in the absence of dendritic cells, was ascertained. Purified NK cells (1×10⁵) were cultured in 96 well-plants, in media that was supplemented with 20 U/ml IL-2 and 10 ng/ml, IL-12, in the absence of, or with increasing concentrations of either activin-A, or TGF-β. Culture supernatants were assayed for IFN-γ after 24 hours of culture, using ELISAs, as described supra.

Activin-A was as potent as TGF-β in inhibiting IFN-γ production by NK cells.

In a second set of experiments, 5×10⁵ purified NK cells were cultured as described herein, in 24 well-plates and were then collected, and lysed after 16 hours of culture.

The lysates were treated to extract the mRNA, which was then subjected to qRT-PCR to determine expression of each of T-bet (a transcription factor), IFN-γ, perforin, granzyme A, and granzyme B.

Activin-A inhibited expression of T-bet and IFN-γ significantly, but had no effect on the expression of the other genes.

With respect to testing viability, 1×10⁵ purified NK cells were cultured as described supra, in 96 well-plates. After 24 hours of culture, cells were stained with antibodies to polycaspase, caspase 3/7, caspase 8 and PI. The staining pattern showed that there was no indication of apoptosis caused by either activin-A or TGF-β.

### Example 20

In these experiments, the effect of activin-A on IFN-γ production by NK cells was tested.

As in the prior experiments, 1×10⁵ purified NK cells were cultured in media supplemented with IL-2 only, or IL-2 plus IL-12, either with or without one of poly I:C or LPS, in the presence or absence of increased levels of activin-A.

The addition of either of poly I:C or LPS to stimulated NK cultures resulted in enhanced IFN-γ production, and this, in turn was suppressed by addition of activin-A, in a concentration dependent manner.

The suppression was, however, reversed when the known activin-A, type I receptor antagonist SB431542, was added, at a concentration of 10 for 30 minutes before the addition of activin-A.

The analysis reported supra was extended to an analysis of the supernatants produced during the various cell culture experiments. A multiplex array system for chemokines and cytokines were used, as was a standard ELISA for measuring IFN-γ. Bead arrays were used to quantify each of IFN-γ, IL-1 IL-6, IL-8, IL-10, TNF-α, GM-CSF, IP-10, MCP-1, MIP-1α, and 1β. Levels of production were measured using standard methodologies.

The experiments confirmed the IFN-γ ELISA data, by showing activin-A's suppressive effect. At 6-12 ng/ml, IFN-γ was the most abundant cytokine produced by the activated NK cells. Activin-A also suppressed the production of IL-6, TNF-α, GM-CSF, and IL-1β by the NK cells, which were the next most abundantly produced cytokines.

It was interesting to note that activin-A increased the low amounts of IL-10 that had been produced, significantly, while suppressing MIP-1β production, as well as MIP-1α, IL-8, and IP-10. The constitutively low amount of MCP-1 produced by activated NK cells, was not affected.

In additional follow up experiments, the NK cells were stimulated with IL-2 or the combination of IL-2 and IL-12, plus poly I:C or LPS, for 16 hours, in the presence of 1 μg/ml of BFA, before a standard ICS assay was carried out via flow cytometry.

A greater percentage of CD56 bright NK cells stained positive for intracellular IFN-γ than did CD56 intermediate populations.

Activin-A did not effect these populations, suggesting that it suppressed the total amount of IFN-γ released, on a per cell basis, rather than altering the number of IFN-γ producing cells.

### Example 21

These experiments impact of activin-A on NK cell CD25 expression.

As above, 1×10⁵ purified NK cells were cultured in 96 well-plates in media that had been supplemented with IL-2 and IL-12, either with or without the addition of TLR ligands, in the presence of activin-A (100 ng/ml).

After 24 hours, supernatants were collected, and stained with anti-CD25 antibodies, with expression being determined by flow cytometry.

CD25 is known as a receptor of the IL-2α chain, and the results indicated that activin-A significantly suppressed the expression of this molecule.

### Example 22

NK cell cytotoxicity is a critical component of immune surveillance.

In these experiments, the effects of activin-A on NK cell killing properties, as well as NKp30 expression, was studied.

1×10⁶ purified NK cells were cultured in 24 well-plates, either with a high dose of IL-2 (100 U/ml), or a combination of 20 U/ml of IL-2 and IL-12. The culturing took place in the absence of either of activin-A or TGF-β or 100 ng/ml of one of them. The culture continued for 3 days.

After the 3 days of culture, the NK cells were combined with target cells K562 or Jurkat cells, at effector (NK cells): target (K562 or Jurkat cells) ratios of 5:1, 2:1, and 0.1:1. The target cells had been labeled with calcein previously. The total number of labeled target cells was 2×10⁴, and the mixture was incubated for 4 hours. The efficiency of killing was determined by measuring calcein dye release via spectrometry.

While TGF-β significantly suppressed the killing ability of NK cells, in striking contrast, activin-A had no impact whatsoever.

In order to investigate the mechanism behind these differences, the same NK cells were purified and cryopreserved immediately after the 3 day culture period. The cells were thawed, washed, and stained for NCR. NKp30, with expression levels determined via flow cytometry. The level of expression was also determined using “ex vivo” samples, and NK cells that were cultured, without exposure to targets.

Culture alone resulted in substantial down regulation of NKp30 as compared to ex vivo cells. Exposure to TGF-β resulted in substantial down regulation of the receptor, while exposure to activin-A had a very limited effect.

Taken as a whole, the results indicate very important biological differences between actions of activin-A and TGF-β on NK cell function. Activin-A suppresses IFN-γ production, but not killing capacity. This is a previously unrecognized regulatory role of activin-A in NK cell function. Clearly, it is distinct from TGF-β and is important in considering the “cross-talk” between dendritic cells and NK cells, in both innate and adaptive immune responses.

The foregoing examples set forth aspects of the invention, which is a method for inhibiting effect of an activin on a cell by adding an amount of an activin inhibitor sufficient to inhibit interaction of said activin on said cell.

The amount of activin inhibitor that is used will, of course, vary, depending upon the context of the use (e.g., in vivo or in vitro, both of which are contemplated), the target cell population, the subject (if in vivo use is being considered), and standard criteria that will be familiar to the skilled artisan. A dose of from about 1 μg to about 500 mg, more preferably from about 10 μg to about 50 mg, and most preferably, from about 100 μg to about 5 mg is preferred. Intravenous administration is preferred, but any form of administration that will successfully place the inhibitor in the immune system is contemplated.

“Activin” as used herein, refers to any member of the activin family, regardless of the species of animal with which that member is associated. Humans, mice, and rats, as well as many other species are known to produce activins, including activin-A which is a homodimer of β_{A} monomers; however, other forms of activin, including but not limited to activin-B, C, and E are included herein.

“Inhibitor” as used herein refers to any substance that prevents interaction of activin with a cell. Any member of the follistatin family, including but not limited to “FS-300”, “FS-288”, and other forms as well. See Hoshimoto, et al., J. Biol. Chem., 272(21):13835-43 (1997) incorporated by reference. There is very high conservation of sequences among both the activins and the follistatins, and it is known, e.g., from the experiments supra, that follistatin from one species will inhibit activin from another, different species. Especially preferred are forms of follistatin which bind to cell surface heparin sulphate proteoglycans, such as FS-288.

Other inhibitors can also be used, such as activin neutralizing antibodies, which are commercially available, as well as substances, such as “SB-431542,” which is commercially available and interacts with activin receptors, thereby impeding interaction of activin with the cells.

It is especially preferred that the inhibitor be contacted with a cell that is considered to be an immune cell which produces activin. As the examples show the addition of activin inhibitory substances to activin producing immune cells acts to increase the potency of those immune cells. Dendritic cells are one type of immune cell which produces activin, and cell type encompassed by this invention, and so are monocytes. It is also known that epithelial cells produce activin-A, and this is also a feature of the invention. Contact may be to any subject, such as, but not being limited to, a mammal, most preferably a human but also domestic animals, including pets, farm animals, and so forth.

Other features of the invention will be clear to the skilled artisan and need not be set forth herein.

The terms and expression which have been employed are used as terms of description and not of limitation, and there is no intention in the use of such terms and expression of excluding any equivalents of the features shown and described or portions thereof, it being recognized that various modifications are possible within the scope of the invention.

1. A method for modifying a CD8⁺ T cell or NK cell response, comprising adding an amount of an activin modulator sufficient to modulate production of activin-A by dendritic cells.

2. The method of claim 1, comprising inhibiting production of activin with an activin inhibitor.

3. The method of claim 1, comprising enhancing production of activin with an activin agonist.

4. The method of claim 2, further comprising enhancing an immune response to an antigen by inhibiting production of activin.

5. The method of claim 1, comprising modulating a CD8⁺ T cell response.

6. The method of claim 1, comprising modulating an NK cell response.

7. The method of claim 1, wherein said activin is activin-A.

8. The method of claim 1, wherein said modulator is follistatin.
